SEQUENCE LISTING (5 sequences)

SEQ ID NO: 1 - DNA, 44 bp, Unknown
  MTB Target
aacctgtggggtccggcctttcacgagaggtatccgaacgtcac

SEQ ID NO: 2 - DNA, 21 bp, Artificial Sequence
  Probe
aacctgtggggtccggccttt

SEQ ID NO: 3 - DNA, 18 bp, Artificial Sequence
  Probe
ttggacaccccaggccgg

SEQ ID NO: 4 - DNA, 20 bp, Artificial Sequence
  Probe
gagaggtatccgaacgtcac

SEQ ID NO: 5 - DNA, 23 bp, Artificial Sequence
  Probe
gtgctctccataggcttgcagtg

(19) United States
(12) Patent Application Publication
(10) Pub. No.: US 2004/0170991 A1
(43) Pub. Date: Sep. 2, 2004

(54) Method for removing amplification inhibitors from test samples

(75) Inventors: Folim G. Halaka, Waukegan, IL (US); Dwight Erickson, Kenosha, WI (US); Qizhi He, Gurnee, IL (US); Gregor W. Leckie, Deerfield, IL (US); Bor-Chian Lin, San Diego, CA (US)

Correspondence Address: STEVEN F. WEINSTOCK, ABBOTT LABORATORIES--D-377/AP6A-1, 100 ABBOTT PARK ROAD, ABBOTT PARK, IL, 60064-5386, US

(73) Assignee: Abbott Laboratories

(21) Appl. No.: 10/463,637

(22) Filed: Jun. 17, 2003

Related U.S. Application Data
(63) Continuation of application No. 08/698,573, filed on Aug. 15, 1996, now abandoned.

(51) Int. Cl. C12Q 1/68

(52) U.S. Cl. 435/6

(57) ABSTRACT

Provided herein is a method for removing nucleic acid amplification reaction inhibitors from test samples.

### FIELD OF THE INVENTION

The present invention relates to nucleic acid amplification reactions and in particular, relates to removing amplification inhibitors from test samples.

### BACKGROUND OF THE INVENTION

Nucleic acid amplification reactions such as the ligase chain reaction (LCR) which is described in European Patent Application EP-A-320-308, the gap ligase chain reaction (GLCR) which is described in U.S. Pat. No. 5,427,930, and the polymerase chain reaction (PCR) which is described in U.S. Pat. Nos. 4,683,202 and 4,683,195 are well known in the art. These and similar reactions have found utility as clinical diagnostic tools to, for example, detect infectious agent in test samples. Amplification reactions have also demonstrated their utility in research and development as well as forensics.

With the number of different areas in which amplification reactions are used comes an equal number of different test samples that contain a nucleic acid sequence of interest (variously referred to as a “target sequence”). Generally, amplification reactions are designed to generate multiple copies of the target sequence and/or the nucleic acid sequence complementary to the target sequence. As previously mentioned, the target sequence can be found in a variety of test samples that may include, for example, blood and blood products such as serum; urine; sputum; cell cultures; and fermentation broths. Unfortunately, in addition to the target sequence, these sources of target sequences often times also carry inhibitors that may impede or prevent amplification of the target sequence. Additionally, given the complexity of a source material itself, as well as its origin, it is difficult to predict whether or not a given source material will have an inhibitor concentration that will impede amplification of a target sequence contained in the source material.

As a result, methods for alleviating the effects of inhibitors on amplification reactions have been devised. Diluting source materials and therefore diluting any inhibitors contained within the source material to a level that is tolerable for amplification is one method for alleviating inhibition. However, such a procedure also dilutes the target sequence and may result in a loss of sensitivity. Test samples have also been centrifuged in an attempt to separate target sequences from their original source material and any inhibitors contained therein. Unfortunately, centrifugation can also separate inhibitors from the original source material along with the target sequences. Affinity purification procedures have also been applied to test samples to separate target sequences from inhibitors but such techniques can require several steps and the reagents employed can be costly. Accordingly, there is a need for a method for removing amplification inhibitors that is effective from a time, cost and effectiveness standpoint.

### SUMMARY OF THE INVENTION

The present invention provides a method for removing amplification reaction inhibitors from test samples that is both time and cost effective. The method can be employed as a sample preparation procedure that is performed prior to amplification of any target sequences that may be in the test sample. The method for alleviating inhibition of nucleic acid amplification comprises acidifying a test sample which will generally comprise a liquid and a target sequence. Upon acidifying the test sample the target sequence is placed in a second liquid that, preferably, has a pH of between about 3.0 and about 4.5. The so-formed second liquid is then replaced with a buffer suitable for nucleic acid amplification. The method is particularly well suited for removing phosphate inhibitors that may complex with ions found in test samples.

### DETAILED DESCRIPTION OF THE INVENTION

While not wishing to be bound by theory, it is believed that many test samples contain phosphate ions as well as calcium ions. These ions can be introduced into the test sample by way of buffers employed to prepare a test sample prior to amplification. Phosphate buffers are one such example. On the other hand, these ions may be indigenous to the test sample itself or they may be present as a result of the combination of ions present in a buffer and ions present in the test sample. When ions such as those exemplified above are present in a neutral to basic solution, they can precipitate out of the solution as calcium phosphate. When this situation arises and target sequences are separated from the original material through for example, centrifugation, the precipitated calcium phosphate is also separated from the original material. Hence, upon suspension in an appropriate buffer, the target sequence as well as the calcium phosphate is resuspended and phosphate ions are released when the calcium phosphate dissolves in the newly formed solution. Thus, when amplification of the target sequence is attempted, the phosphate ions compete with phosphate groups present on amplification primers for active sites on enzymes employed in the amplification reaction. Inhibition of amplification thereby arises.

Applicants have discovered a method for removing inhibitors from a test sample that can be performed in a straight forward manner and, importantly, is effective at removing inhibitors from a test sample to the extent that they no longer inhibit amplification of the target sequence. Basically, the method includes providing a test sample comprising a liquid and a target sequence; acidifying the test sample; and replacing the acidified liquid containing any target sequences with a buffer suitable for nucleic acid amplification. Again not wishing to be bound by theory, it is believed that by acidifying a solution containing the target sequences as well as, for example, calcium and phosphate ions, calcium phosphate is not able to precipitate out of solution and is therefore not co-separated with the target sequence when the target sequence is placed in a buffer suitable for nucleic acid amplificaiton. Thus, inhibitors are removed and when amplification of the target sequence is performed, inhibition is relieved.

The term “test sample” as used herein means anything suspected of containing the target sequence. The test sample can be from any biological source, such as for example, blood, bronchial alveolar lavage, saliva, throat swabs, ocular lens fluid, cerebral spinal fluid, sweat, sputa, urine, milk, ascites fluid, mucous, synovial fluid, peritoneal fluid, amniotic fluid, tissues such as heart tissue and the like, or fermentation broths, cell cultures, chemical reaction mixtures and the like. The test sample can be used (i) directly as obtained from the source or (ii) following a pre-treatment to modify the character of the sample. The test sample can be pre-treated prior to use by, for example, preparing plasma from blood, disrupting cells, preparing liquids from solid materials, diluting viscous fluids, filtering liquids, distilling liquids, concentrating liquids, adding reagents, and the like. While pretreatment steps may change the nature of the test sample as obtained from the source, the target sequence may nevertheless be contained within the derived product and therefore the derived product remains a test sample.

A subset of the term test sample is a “clinical test sample” which is a test sample in that it will be suspected of containing a target sequence, but it is also a sample that has undergone minimal pretreatment or purification and is essentially as taken from the source. Examples of clinical test samples include, but are not intended to be limited to sputum, whole blood, serum, plasma and urine. Thus, samples which have undergone crude separation techniques, such as filtration and centrifugation are not excluded from the term clinical test sample provided they otherwise meet the definition of that term given above.

A “target sequence” as used herein means a nucleic acid sequence that is or will be detected, amplified, or both amplified and detected. While the term target sequence is sometimes referred to as single stranded, those skilled in the art will recognize that the target sequence may actually be a double stranded sequence comprising a nucleic acid sequence and its complement. Often times, a target sequence will be found in a cell such as a bacterium.

As used herein the term “acidifying” means changing the pH of a test sample to less than 7.0, typically between about pH 2.0 and about pH 5.5, and more typically between about pH 3.0 and about pH 4.5. Methods for acidifying a test sample are well known in the art and may include adding any well known acid directly to a test sample, or separating the target sequence from an original source and placing it in an acidic buffer. Acidic buffers can be any aqueous solution comprising a buffering system and having a pH in the ranges specified above. Buffering systems are known in the art and generally comprise aqueous solutions of compounds which resist changes in the hydrogen ion concentration of the solution. Hence, their primary function is to maintain a desired pH. Examples of buffering systems include, but are not intended to be limited to solutions of a weak acid or base and salts thereof such as, for example, acetates, borates, phosphates, phthalates, citrates, carbonates and the like. Typically an acetate buffering system is used having a molarity of between about 25 mM and about 2 M, more typically between about 100 mM and about 1 M and most typically between about 250 mM and about 1 M.

The term “replacing” as used herein, in particular as used with respect to replacing one buffer or solution with another buffer or solution, means separating target sequences from one buffer or solution and placing them in another buffer or solution. Many means for accomplishing such a replacement are well known and a matter of choice for one skilled in the art. Techniques for achieving such an exchange include but are not limited to dialysis, size exclusion and ion exchange chromotography with resins, as well as centrifugation and suspension of a pellet formed as a result of the centrifugation.

As previously mentioned, the present method is well suited for preparing test samples prior to running an amplification reaction. Amplification reactions are well known and include but are not limited to Amplification reactions such as, for example, LCR, GLCR and PCR are well known in the art. These reactions typically employ primers to repeatedly generate copies of a target nucleic acid sequence which is usually a small region of a much larger nucleic acid sequence. Primers and probes are themselves nucleic acid sequences that are complementary to regions of a target sequence and under amplification conditions, hybridize or bind to the complementary regions of the target sequence. Copies of the target sequence are typically generated by the process of primer extension and/or ligation which utilizes enzymes with polymerase or ligase activity, separately or in combination, to add nucleotides to the hybridized primers and/or ligate adjacent primer pairs. The nucleotides that are added to the primers or probes, as monomers or preformed oligomers, are also complementary to the target sequence. Once the primers or probes have been sufficiently extended and/or ligated they are separated from the target sequence, for example, by heating the reaction mixture to a “melt temperature” which is one where complementary nucleic acid strands dissociate. Thus, a sequence complementary to the target sequence is formed. A new amplification cycle can then take place to further amplify the number of target sequences by separating any double stranded sequences, allowing primers to hybridize to their respective targets, extending and/or ligating the hybridized primers and re-separating. The complementary sequences that are generated by amplification cycles can serve as templates for primer or probe extension to further amplify the number of target sequences.

In addition to the enzymatic thermal amplifications described above, isothermal enzymatic amplification reactions could also be employed according to the instant invention. For example, “3SR” (Self-Sustained Sequence Replication) described in Fahy, E., et. al., PCR Methods and Applications, 1: 25-33 (1991) and “SDA” (Strand Displacement Amplification) described in Walker, G. T., et. al., PNAS 89: 392-396 (1992).

Reagents for performing these reactions are well known and include but are not limited to: enzymes such as polymerase, ligase and reverse transcriptase; enzyme cofactors such as magnesium; salts; nicotinamide adenine dinucleotide (NAD); and deoxynucleotide triphosphates (dNTPs) such as for example deoxyadenine triphosphate, deoxyguanine triphosphate, deoxycytosine triphosphate and deoxythymine triphosphate. These reagents are generally added to a buffer suitable for nucleic acid amplification reactions. EPPS, which refers to N-(2-hydroxyethyl) piperazine-N′-(3-propanesulfonic acid), buffer is an example of one such well known buffer.

According to one embodiment, the method comprises the steps of providing a liquid test sample, acidifying the test sample to thereby place any target sequences in an acidic solution; and replacing the acidified solution containing the target sequences with a buffer suitable for nucleic acid amplification reactions.

According to another embodiment, the method is performed after the original test sample is pretreated with a basic solution (i.e. pH greater than 7.0, more typically greater than about pH 8.0). For example, sputum is often times taken as a test sample for nucleic acid amplification based diagnostic methods designed to detect Mycobacterium tuberculosis (MTB). In order to inactivate micororganisms other than MTB in the test sample prior to culture analysis, a solution of sodium hydroxide is added to the sample and the sample is incubated for an appropriate time before a phosphate buffer is added to the solution. According to this embodiment, after treatment with the basic solution, the solution is acidified prior to replacing the acidified solution with a buffer suitable for nucleic acid amplification reactions. According to this embodiment, the basic solution can be acidified by, for example, adding acid or an acidic buffer directly to the basic solution until the pH is between about 2.0 and about pH 6.0 before the acidic solution, containing any target sequences, is replaced with a buffer suitable for nucleic acid amplification.

According to another embodiment, a clinical test sample such as sputum is contacted with sodium hydroxide and phosphate buffer to form a basic test sample. The basic test sample is incubated at room temperature for about 15 minutes before it is centrifuged at a speed sufficient to form a pellet. The pellet is first suspended in saline and a portion thereof is then acidified an about 50 mM to about 1 M acetate buffer having a pH between about pH 3.5 and about pH 4.5 to form a second test sample. The second test sample is then centrifuged under conditions sufficient to form a pellet and the pellet is resuspended in a buffer suitable for nucleic acid amplification.

The following examples are provided to further illustrate the present invention and not intended to limit the invention.

### EXAMPLES

In the following examples, the present method is shown to alleviate the effects of inhibitors on nucleic acid amplification. In all of the examples gap LCR, as described in U.S. Pat. No. 5,427,930, was employed to amplify a MTB target sequence (SEQ. ID. NO. 1) using four probes designated SEQ. ID. NO. 2, SEQ. ID. NO. 3, SEQ. ID. NO. 4, and SEQ. ID. NO. 5. Opposite ends of adjacent probes were labeled with carbazole or adamantane haptens as described in U.S. Pat. Nos. 5,424,414 and 5,464,746. Haptenated amplification products were detected with an LCx® analyzer (Abbott Laboratories, Abbott Park, Ill.) using anti-carbazole antibody coated microparticles and an anti-adamantane antibody conjugated with alkaline phosphatase.

### Example 1

### Simulation of CaHPO₄ Effects on Amplification

In this example, a solution of CaCl₂ was used as a test sample and demonstrates the effects that an ion such as calcium (an ion that can be found in clinical samples) can have on nucleic acid amplification reactions in the presence of phosphate.

10 ml of 5 mM CaCl₂ was mixed with 10 ml of 3% (w/v) NaOH and incubated for 15 minutes. After incubation of the NaOH treated CaCl₂ solution, 30 ml of 67 mM sodium phosphate buffer (pH 6.8) was added to the solution and the resulting solution was centrifuged at 2000×g for 30 minutes. The supernatant was discarded and the pellet was resuspended in 0.5 ml TE buffer [10 mM tris(hydroxymethyl) aminomethane (Tris®), 1 mM ethylenediamene tetraacetic acid (EDTA), pH 8.0]. 30 μl of glass beads were then added to the tubes. The above procedure was performed in triplicate in three different microfuge tubes. 0.9 ml of 75 mM EPPS buffer pH 8.0 [EPPS refers to N-(2-hydroxyethyl) piperazine-N′-(3-propanesulfonic acid)] was then added to one microfuge tube. 0.9 ml of 500 mM sodium acetate buffer (pH 4.1) was added to a second microfuge tube and 0.9 ml of 1 M sodium acetate buffer (pH 4.1) was added to the last microfuge tube.

The three microfuge tubes containing the NaOH treated CaCl₂ and the various buffers were then centrifuged at 2000×g for 10 minutes. The supernatants were aspirated and the pellets (approximately 200 μl) were resuspended in 1 ml of TE buffer. Centrifugation was repeated and the pellets were resuspended in 400 μl of TE buffer. The tubes were then boiled for 15 minutes, and sonicated for 10 minutes at 40 Watts of power and 37 kHz frequency. 10 μl of H37Ra MTB DNA (apporximately 25 molecules) were then added to 100 μl of the sonicated liquid from each of the tubes. The DNA containing solutions were then added to a unit dose amplification mixture containing the following: 40 μl 100 mM EPPS, 0.2 μl 0.5 M EDTA, 0.5 μl 1 mM nicotinamide adenine dinucleotide (NAD), 0.34 μl 1 mM d-adenosine 5′ triphosphate (dATP), 0.34 μl 1 mM d-cytidine 5′ triphosphate (dCTP), 0.2 μl 10% sodium azide, 10¹² molecules of each of the probes, 0.133 μl of 1.5 M spermidine, 18,000 units of ligase enzyme, 2 units of polymerase enzyme, and 56.7 μl distilled deionized water. The mixtures were then subjected to 37 cycles of programmed temperature change, with each cycle being 94° C. for 1 second, 64° C. for 1 second and 69° C. for 40 seconds. The thermal cycling was performed in a PE480 thermal cycler available from Perkin-Elmer, Norwalk, Conn. The amplification products were then detected using the LCx® analyzer (Abbott) and the results are shown in Table 1.

TABLE 1

| Resuspension Buffer | LCx ® Rate |
|---|---|
| 1 M Acetate pH 4.1 | 1293 |
| 500 mM Acetate pH 4.1 | 1083 |
| 75 mM EPPS | 23 |

As shown by the results in Table 1, acidifying the initial pellet by resuspending it in an acidic buffer permitted efficacious amplification of the target sequence. On the other hand, amplification did not effectively occur in the sample that did not receive acid treatment.

### Example 2

### Amplification of MTB DNA With and Without Acid Treatment

In this example, phosphate inhibition and relief of phosphate inhibition with the present method is demonstrated using simulated calcium phosphate precipitate.

The inhibitory precipitate was prepared by mixing 10 ml of 50 mM Calcium Chloride with 10 ml of 3% NaOH and adding 30 ml of 67 mM phosphate buffer. A control using no calcium chloride was also prepared. These samples were centrifuged (3000×g for 15 minutes) and the supernatant was decanted leaving about 5 ml. 5 ml of TE buffer was added to suspend the 5 ml sample pellets. 0.25 ml of these samples were then added to 1.25 ml of 300 mM acetate buffers having pH's of 4.1, 4.4, 4.7, and 5.0. After centrifuging these samples the supernatants were removed and the pellets were suspended in 0.5 ml of TE buffer. 90 μL of each sample and 10 μL of MTB DNA (25 molecules) were then placed in the amplification mixture, amplified and detected in the same manner set forth in example 1. The amplification results are shown in Table 2.

TABLE 2

LCx ® Reading

| Buffer | No CaCl₂ | 0.05 M CaCl₂ |
|---|---|---|
| Acetate pH 4.1 | 532 | 591 |
| Acetate pH 4.4 | 631 | 547 |
| Acetate pH 4.7 | 631 | 696 |
| Acetate pH 5.0 | 630 | 581 |
| TE pH 8.0 | 639 | 20 |

As shown by Table 2, amplification was effective in all samples treated with CaCl2 that were acidified according to the present method. On the other hand, the tube that was treated with CaCl₂ but not acidified (TE pH 8.0) demonstrated amplification inhibition.

### Example 3

### Inhibition Relief Using Clinical Samples

In this example, 50 clinical sputum samples were tested according to the present method.

The samples were collected from a clinical microbiology laboratory and had previously been prepared using a clinical procedure for preparing sputum samples that comprises numerous steps of buffer additions and removals. The first step is to combine equal volumes of sputum and a 2% sodium hydroxide solution before incubating for 15 minutes at room temperature. This step is often used to inactivate microorganisms other than MTB contained in normal sputum. After the prescribed incubation time a 67 mM phosphate buffer (pH 6.8) is added to the NaOH decontamination solution. The entire sample is then centrifuged and the supernatant poured off and discarded. The remaining pellet, which contains the MTB cells, and thus the target sequences, is suspended in a neutral saline solution. 0.25 ml of each sample was added to 1 replicate each of 1.25 ml of TE buffer and 1.25 ml of 300 mM acetate buffer pH 4.1. The samples were centrifuged (10 min. 1500×g), the supernatants were removed, and the resulting pellets were suspended in 0.5 ml of EPPS buffer. The samples were then heated in a boiling water bath for 15 minutes to inactivate the MTB and sonicated for 10 minutes at 40 watts of power and 37 kHz frequency. 100 μL of sample and 10 μL of MTB DNA (25 molecules) were then placed in the amplification mixture, amplified and detected in the same manner set forth in example 1. The amplification results are shown in Table 3.

TABLE 3

LCx ® Rate

| Sample | TE Buffer | Acetate Buffer pH 4.1 |
|---|---|---|
| 1 | 31 | 759 |
| 2 | 700 | 518 |
| 3 | 1000 | 679 |
| 4 | 10 | 616 |
| 5 | 460 | 817 |
| 6 | 317 | 755 |
| 7 | 76 | 617 |
| 8 | 14 | 724 |
| 9 | 649 | 489 |
| 10 | 675 | 529 |
| 11 | 183 | 714 |
| 12 | 456 | 679 |
| 13 | 458 | 794 |
| 14 | 783 | 514 |
| 15 | 591 | 530 |
| 16 | 182 | 484 |
| 17 | 694 | 318 |
| 18 | 620 | 743 |
| 19 | 836 | 585 |
| 20 | 712 | 815 |
| 21 | 997 | 814 |
| 22 | 802 | 683 |
| 23 | 1063 | 719 |
| 24 | 876 | 801 |
| 25 | 880 | 908 |
| 26 | 904 | 795 |
| 27 | 1194 | 851 |
| 28 | 17 | 1163 |
| 29 | 852 | 819 |
| 30 | 846 | 860 |
| 31 | 1082 | 786 |
| 32 | 859 | 508 |
| 33 | 1009 | 753 |
| 34 | 1024 | 1011 |
| 35 | 1006 | 85 |
| 36 | 856 | 988 |
| 37 | 113 | 715 |
| 38 | 11 | 953 |
| 39 | 913 | 725 |
| 40 | 842 | 661 |
| 41 | 757 | 659 |
| 42 | 9 | 743 |
| 43 | 8 | 972 |
| 44 | 8 | 920 |
| 45 | 485 | 876 |
| 46 | 946 | 733 |
| 47 | 827 | 802 |
| 48 | 608 | 946 |
| 49 | 890 | 864 |
| 50 | 945 | 859 |

Having been spiked with the target sequence, all samples should have had a positive result. As shown by Table 3, samples 1, 4, 7, 8, 11, 16, 28, 37, 38, 42, 43 and 44 demonstrated the effects of inhibition as they yielded a negative result in the absence of treatment according to the present method. However, the same samples treated according to the present method yielded a positive result which demonstrates the effectiveness of the invention has been the present method at alleviating inhibition.

While invention has been described in detail and with reference to specific embodiments, it will be apparent to one skilled in the art that various changes and modifications may be made to such embodiments without departing from the spirit and scope of the invention

What is claimed is:

1. A method for alleviating inhibition in nucleic acid amplification assays comprising the steps of:
a) providing a test sample comprising a liquid and a target sequence;
b) acidifying said test sample to place said target sequence in a second liquid; and
c) replacing said second liquid with a buffer suitable for nucleic acid amplification.

2. The method of claim 1 wherein prior to step b), said target sequence is contacted with a basic phosphate containing solution.

3. The method of claim 2 wherein said test sample is a clinical test sample.

4. The method of claim 1 wherein said test sample further comprises calcium ions.

5. The method of claim 1 wherein said acidifying comprises:
a) centrifuging said test sample to form a supernatant and a pellet;
b) removing said supernatant; and
c) resuspending said pellet in an acidic buffer.

6. The method of claim 5 wherein said acidic buffer comprises between 100 mM and 2 M sodium acetate and wherein said buffer has a pH of between 3.0 and 4.5.

7. In a method for preparing a test sample for nucleic acid amplification comprising the steps of:
a) treating said sample with a basic solution to thereby form a basic test sample; and
b) placing any target sequences contained in said test sample into a buffer suitable for nucleic acid amplification;
wherein the improvement comprises acidifying said basic test sample after step a) and before step b).

8. The method of claim 7 wherein said test sample is a clinical test sample.

9. The method of claim 8 wherein said method further comprises treating said basic solution with a phosphate containing solution prior to step b).

10. The method of claim 7 wherein said acidifying comprises:
a) centrifuging said basic test sample to form a supernatant and a pellet;
b) removing said supernatant; and
c) resuspending said pellet in an acidic buffer.
